(19) United States
(12) Patent Application Publication
(10) Pub. No.: US 2019/0380635 A1
(43) Pub. Date: Dec. 19, 2019

(54) Dual-Sided Biomorphic Polymer-based Microelectrode Array and Fabrication Thereof

(71) Applicant: Greg Gerhardt, Sunnyvale, CA (US)

(72) Inventor: Greg Gerhardt, Sunnyvale, CA (US)

(73) Assignee: Greg Gerhardt, Sunnyvale, CA (US)

(21) Appl. No.: 16/234,570

(22) Filed: Dec. 28, 2018

Related U.S. Application Data
(62) Division of application No. 15/269,769, filed on Sep. 19, 2016, now abandoned.

(51) Int. Cl. A61B 5/1486; A61N 1/05; A61B 5/00

(52) U.S. Cl. CPC A61B 5/14865; A61N 1/0531; A61B 2562/043; A61B 2560/02; A61B 2562/12; A61B 5/4064

(57) ABSTRACT

A dual-sided biomorphic polymer-based microelectrode array and method of fabricating the same. A measurement probe fabricated from a polymer consisting of two sides each with an array of paired recording sites for the measurement of molecules in an aqueous biological or chemical environment. Enzyme-based coatings are placed on microelectrodes of one measurement probe side specific to analytes of interest, and are coupled with a similar but non-functional protein matrix coating on the microelectrode on the opposite side to yield two distinct recording sites for subtraction of interferents, noise and non-Faradaic background current. Microelectrodes are arranged with variable spacing between each to match a variety of brain structures affording a biomorphic array allowing simultaneous recordings at multiple target depths and coordinates from one measurement probe system. The fabrication method uses photolithographic techniques where each dual-sided biomorphic polymer-based microelectrode array is cut out using lithography, allowing for multiple different or identical designs that can be simultaneously patterned on a single polymer wafer and improved microelectrode tip that is tapered for improved tissue penetration.

### CROSS REFERENCE TO RELATED APPLICATIONS

This application is a divisional of application Ser. No. 15/269,769 filed Sep. 19, 2016 which claims priority under 35 U.S.C. § 119 (e) to U.S. provisional patent application Ser. No. 62/219,671 filed on Sep. 17, 2015, the contents of which is hereby incorporated herein by reference in its entirety for all purposes.

### BACKGROUND OF THE INVENTION

The present invention relates generally to electrochemical recordings in biological systems and more specifically dual-sided biomorphic bioflex polymer-based microelectrode arrays and a method of fabrication of the same.

The detection of neurotransmitters and metabolic molecules requires reproducible and biologically inert microelectrode configurations using materials that will handle the high ionic strength and protein environments of central nervous system tissues. Historically, electrochemical recordings of neurotransmitters (including glutamate, acetylcholine, choline, GABA, adenosine, dopamine, norepinephrine, and serotonin) and metabolic molecules (including oxygen, glucose, lactate, pyruvate, and ATP) have focused on ways to minimize the “non-Faradaic” or non-specific background signals such as solvent dipole reorientation, adsorption, desorption, and movement of electrolyte ions at the recording site surfaces from measurements such that the major signal measured is due to the analyte of interest. In biological systems there are many contributors to changes in an electrode's background, or non-Faradaic response, such as pH shifts and changes in Ca²⁺, Na⁺, or Cl⁻ ions.

Self-referencing has been an extremely powerful tool for removing the effects of chemical interferents that might contribute to a portion of the analyte signal and for permitting the subtraction of noise, which is present on both the analyte and control sites, from the analyte signal. One of the biggest advantages of self-referencing is that it makes it possible to measure both tonic levels and phasic changes of the neurochemical, rather than only the changes from a baseline or transient neurotransmitter changes. However, self-referencing subtraction cannot be achieved using single microelectrodes.

Fabrication of microelectrodes using the basic concepts of photolithographic formation of microelectrodes on silicon substrates was used in the development of ceramic, substrate-based biomorphic electrode arrays (bMEAs), allowing for single-sided recording sites. However, while ceramic bMEAs have proven useful, there remain a number of issues surrounding the use of the bMEAs in biological systems such as their fragility, limitations of post lithographic cutting, the complexity of dual-sided configurations and the size limitations of ceramic substrates.

It is within the aforementioned context that a need for the present invention has arisen. Thus, there is a need to address one or more of the foregoing disadvantages of conventional systems and methods, and the present invention meets this need.

### BRIEF SUMMARY OF THE INVENTION

Various aspects of dual-sided biomorphic polymer-based microelectrode arrays and fabrication thereof can be found in exemplary embodiments of the present invention.

In a first embodiment, a biomorphic microelectrode array (bMEA) allows chemical and metabolic studies in multiple brain sub-regions simultaneously without moving the recording device up and down as well as the dual matching front and back recording sites for self-referencing recording described below [08]. Multiple recording sites allow for tonic and phasic recordings of neurochemical and metabolic molecules that cannot be measured by single microelectrodes. The multisite aspect of the sensor provides for examination of neurotransmitter system function at multiple brain locations and within brain structures.

In another embodiment, microelectrodes are coated to have sentinel and analyte-sensing sites in close, defined proximity. A dual-sided bMEA with identical recording sites on both the front and back of the microelectrode has one side coated with enzymes for detection of a specific analyte such as glutamate or glucose, lactate and a variety of other molecules, and the reverse side is coated as a control for self-referencing. Recording surfaces of matching platinum (Pt) crystal structure produce similar responses of catalyzed oxidation and/or reduction of reporter molecules enhancing the signal-to-noise of these microelectrodes for brain recordings. A front-back design allows for optimum self-referencing subtraction of non-Faradaic background signals, elimination of noise and subtraction of unknown chemical interferents.

In a further embodiment, bMEAs are mass fabricated using photolithographic techniques and cut out using lithography allowing for the formation of a low insertion force tip unlike prior designs. Hundreds of bMEAs with different or identical designs can be simultaneously patterned on a single polymer wafer. High purity Pt (0.25 μm) is sputtered onto the recording sites. bMEAs with 4-8 recording sites have been fabricated with recording sites ranging from 10×10 μm to 50×300 μm. Various recording site sizes and arrangements can be selected to conform to specific brain layers. The front and back of the polymer substrate are patterned to increase recording site density and create isolated front and back site recording pairs, where if one site or pair fails useful data can be received from the other recording sites.

A further understanding of the nature and advantages of the present invention herein may be realized by reference to the remaining portions of the specification and the attached drawings. Further features and advantages of the present invention, as well as the structure and operation of various embodiments of the present invention, are described in detail below with respect to the accompanying drawings. In the drawings, the same reference numbers indicate identical or functionally similar elements.

### BRIEF DESCRIPTION OF THE DRAWINGS

FIG. 1A illustrates a microelectrode probe system according to an exemplary embodiment of the present invention.

FIG. 1B illustrates a microelectrode site cross section within an exemplary probe system according to an exemplary embodiment of the present invention.

FIG. 1C illustrates a biomorphic multi-site microelectrode array within an exemplary probe system according to an exemplary embodiment of the present invention.

FIG. 2 illustrates a profile of a dual-sided biomorphic polymer-based microelectrode array in accordance with an exemplary embodiment of the present invention.

FIG. 3 illustrates enzyme-based coatings of microelectrodes in accordance with an exemplary embodiment of the present invention.

FIG. 4 illustrates a process for fabricating dual-sided biomorphic polymer-based microelectrode arrays in accordance with an exemplary embodiment of the present invention.

### DETAILED DESCRIPTION OF THE INVENTION

Reference will now be made in detail to the embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth to provide a thorough understanding of the present invention. However, it will be obvious to one of ordinary skill in the art that the present invention may be practiced without these specific details. In other instances, well-known methods, procedures, components, and circuits have not been described in detail as to not unnecessarily obscure aspects of the present invention.

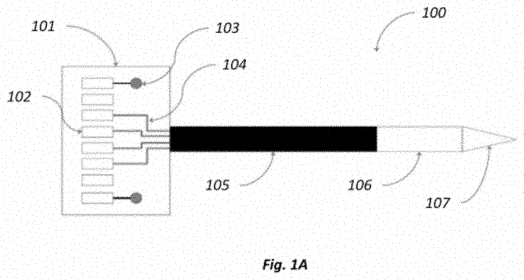

FIG. 1A illustrates a microelectrode probe system 100 according to an exemplary embodiment of the present invention.

In FIG. 1A, microelectrode probe system 100 comprises, among other components, interface 101 containing conductive contacts 102 connected with communication channels 104 and other contacts 103. Interface 101 is physically coupled with probe body 105 comprising housing 106 and tip 107, both containing other probe components. Communication channels 104 can be any medium that allows communication from one point to another, for example for transmitting signals from components within probe body 105 to conductive contacts 102. As shown, microelectrode probe system 100, probe body 105, housing 106, and tip 107 may be composed of a single physical layer or a combination of a plurality of physical layers.

Probe body 105, housing 106, and tip 107 may be rigid or flexible. Microelectrode probe system 100 is used to measure chemical and electrical values using components within probe housing 106 and probe tip 107. Although not shown, other devices may be connected to microelectrode probe through interface 101 and conductive contacts 102 to retrieve information from probe components through communication channels 104. Components within the probe housing 106 and probe tip 107 can include any type of sensor or network of sensors appropriate for the desired physiological measurements to be collected.

FIG. 1B illustrates a microelectrode site cross section 110 within a probe system according to an exemplary embodiment of the present invention. It will be appreciated that microelectrode site cross section 110 as depicted in FIG. 1B is illustrative of a cross section of an exemplary probe system, similar to the example provided by FIG. 1A.

In FIG. 1B, microelectrode site cross section 110 within an exemplary probe system comprises probe body 105 and housing 106 containing communication channels 104 individually coupled with a microelectrode 111. It will be appreciated that communication channels 104 within housing 106 can be communicably coupled with communication channels 104 within interface 101 as depicted in FIG. 1A. Probe body 105 and housing 106 may be rigid or flexible. Communication channels 104 can be any medium that allows communication from one point to another for example for transmitting signals from microelectrode 111 within probe body 105 to conductive contacts 102 within interface 101 as depicted in FIG. 1A. Microelectrode 111 performs measurement of characteristics of a substance and communicates measurement information through communication channel 104. As shown, microelectrode site cross section 110 within an exemplary probe system, probe body 105, and housing 106 may be composed of a single layer or a combination of a plurality of physical layers.

FIG. 1C illustrates a biomorphic multi-site microelectrode array within a probe system 120 according to an exemplary embodiment of the present invention.

In FIG. 1C, biomorphic multi-site microelectrode array within an exemplary probe system 120 comprises, among other things, probe body 105, housing 106, and tip 107. Probe body 105, housing 106, and tip 107 may be rigid or flexible. Housing 106 and tip 107 contain a plurality of microelectrodes 111 each coupled with an individual communication channel 104.

Microelectrode 111 may be placed at variable locations within housing 106 in order to achieve specific measurements at a given probe location. Biomorphic multi-site microelectrode array within a probe system 120 contains a plurality of microelectrodes 111 of no fixed amount. Placement and spacing of microelectrodes 111 in a linear and/or paired arrangement (100 microns to 3 mm) is designed to match a variety of brain structures affording a biomorphic array allowing simultaneous recordings at multiple target depths and coordinates from one microelectrode probe system 100.

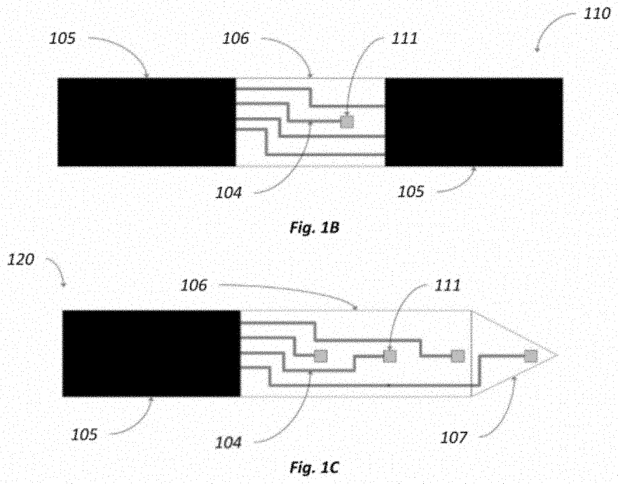

FIG. 2 illustrates a profile of a dual-sided biomorphic polymer-based microelectrode array 200 according to an exemplary embodiment of the present invention.

In FIG. 2, dual-sided biomorphic polymer-based microelectrode array 200 comprises an upper side 200A and a lower side 200B, the upper and lower sides separated by polymer and/or other material layer 201, each side containing interface 101 physically coupled with flexible probe body 105 comprising housing 106 and tip 107.

Housing 106 and tip 107 contain a plurality of microelectrodes (not shown, although examples are depicted in the preceding Figures, for example microelectrode 111) each connected to an individual communication channel (not shown, although examples are depicted in the preceding Figures, for example communication channel 104). It will be appreciated that, while probe body 105 may appear to be connected to and not containing house 106 and tip 107 in the Figures herein, probe body 105 comprises both house 106 and tip 107.

According to one embodiment, an individual microelectrode (not shown) 111 on one side of dual-sided biomorphic polymer-based microelectrode array 200 is responsible for measurement of a specific analyte such as glutamate or glucose, lactate and a variety of other molecules. Each microelectrode 111 is paired with a corresponding microelectrode 111 on the reverse side of dual-sided biomorphic polymer-based microelectrode array 200. The corresponding microelectrode 111 is responsible for detection of a specific analyte and is used as a control for self-referencing. Enzyme-based coatings on microelectrodes 111 on one side specific to analytes of interest (active), coupled with a similar but non-functional protein matrix (sentinel) coating on the microelectrode 111 on the opposite side yields two distinct recording sites for subtraction of interferents, noise and non-Faradaic background current. Differences in the measurement recorded on paired microelectrodes 111 on each side of the dual-sided biomorphic polymer-based microelectrode array 200 provide for the identification and elimination of interfering signals. Dual-sided biomorphic polymer-based microelectrode array 200 allows for improved signal to noise affording tonic and phasic recordings of neurochemical and metabolic molecules that cannot be measured by single microelectrodes. The disclosures of Burmeister and Gerhardt, Analytical Chem 2001, 73 and Miller et al, J Neuroscuience Methods, 252 (2015) provide explanation of self-referencing and improved signal to noise as referenced herein; the disclosures of both publications are hereby incorporated by reference in their entirety.

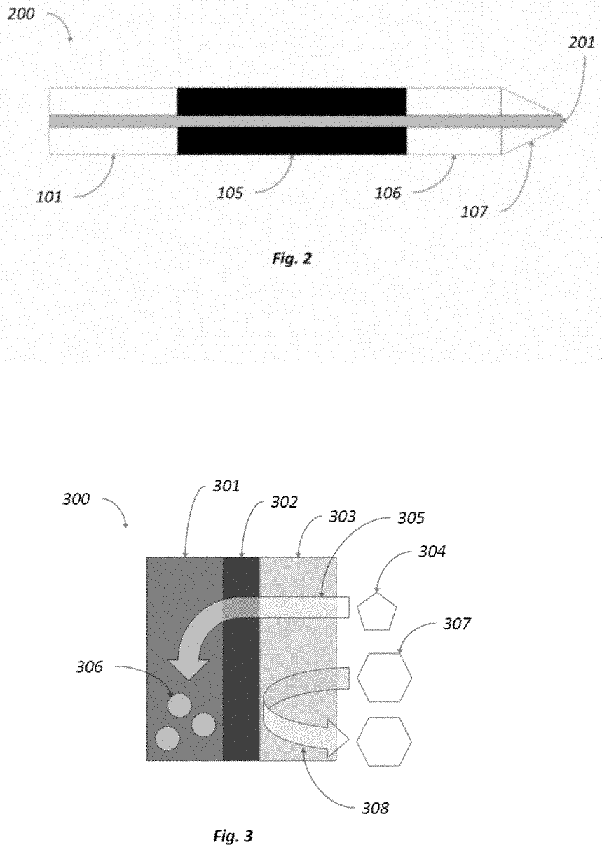

FIG. 3 illustrates enzyme-based coatings of microelectrodes 300 in accordance with an exemplary embodiment of the present invention.

In FIG. 3, enzyme-based coatings of microelectrodes 300 comprise three layers. Communication layer 301 is separated from enzyme layer 303 by a barrier layer 302. Communication layer 301 may be comprised of a sputtered platinum (Pt) metal layer to produce catalyzed oxidation of molecules (or reduction depending on analyte measured) 306 for reporting measurements. Barrier layer 302 may be comprised of poly-(meta-phenylenediamine) (mPD) to minimize the oxidization of unwanted molecules applied to the communication layer 301. Enzyme layer 303 comprises an enzyme coating specific or highly selective to an analyte to be measured. Enzyme layer 303 is also referred to as an enzyme coating on the recording site.

When the enzyme layer 303 is coated with a specific enzyme such as, but not limited to, glutamate oxidase, allowed molecules 304 such as glutamate interact and pass 305 reporter molecules such as but not limited to H₂O₂ 306 to the communication layer 301. Other molecules 307 cannot either interact with the selective enzyme layer and/or are prevented from passing 308 certain oxidizable and/or reducible molecules to the communication layer 301 by the barrier layer 302.

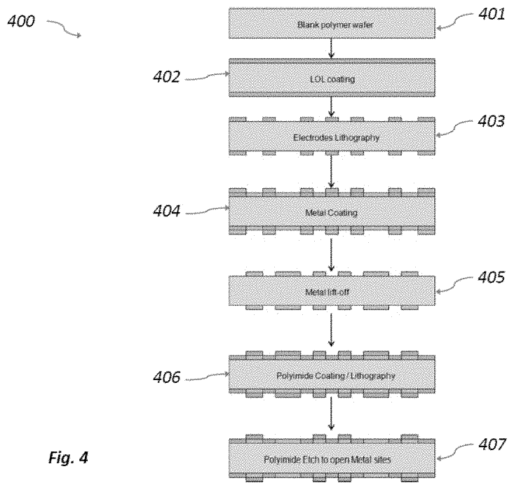

FIG. 4 illustrates a process 400 for fabricating dual-sided biomorphic polymer-based microelectrode arrays in accordance with an exemplary embodiment of the present invention.

In FIG. 4, a microfabrication process 400 starts with flexible polymer wafer 401 such as laminated Kapton blank polymer wafer or acrylic substrate. Lift-off layer (LOL) is then spun 402 onto the wafer and pre-baked.

In a next lithography step 403, the LOL is exposed to light in a mask aligner and etched to open areas for TiPt metal deposition.

In a metal coating step 404, high purity Pt (0.25 μm) is sputtered conformally. The Pt on LOL is then lifted and removed 405, and Pt on polymer substrate is left on the polymer wafer surface forming the desired device pattern.

Next the wafer undergoes a polyimide coating and lithography step 406. The wafer with Pt pattern is conformally spin-coated with polyimide layer (0.2 μm), and pre-baked. The wafer is then coated with photo resist (PR), baked, and exposed to the light in a mask aligner.

Finally, the wafer is etched 407 in a developer to open areas for etching polyimide and finish formation of electrical insulating coating on conducting traces. The etching defines precisely the areas of recording sites of the electrodes.

According to one embodiment, the process 400 described in FIG. 4 includes a high definition lift-off process for thicker Ti/Pt deposition (e.g., greater than 0.16 μm). The lift-off process is optimized to minimize Ti/Pt lifting at the contour of breaking through the metal. A thicker Ti/Pt layer enables reliable wire bonding and soldering of electrical connectors. This provides more options in electrical connector selection and packaging types.

According to one embodiment, sputtering is used in the process 400 described in FIG. 4 for the benefit of lower temperatures as compared to those when e-beam evaporation is used, and the sputtering is done in stages for controlling temperatures below 90 degrees Celsius on the substrate.

According to one embodiment, the process 400 described in FIG. 4 is applicable to both rigid substrates (alumina—Al2O3) and flexible substrates (Kapton—polyimide, acrylic, etc.). Rigid substrates are more limited in size as compared to flexible substrates which can be substantially larger in size. Flexible substrates and rigid substrates differ in packaging in that, in the case of flexible substrates, it can be easier to attach connectors and/or create connectors by soldering pins to the substrate.

The process 400 described in FIG. 4 can also be used on other types of substrates such as acrylic (PMMA), silicon (Si) and the like.

According to one embodiment, thin polyimide and/or SU-8 conformal coating applied over the conducting traces serves for electric insulation purposes and in some designs to define geometry of electric recording sites. Long (on the scale of 100 mm or longer) and thin (less than 0.25 mm×0.25 mm cross section) polyimide flexible probes minimize damage and inflammation in brain tissue.

According to one embodiment, a stainless steel wafer is used in process 400. The polymer layer(s) are then be layered on each side of the stainless steel wafer, enabling creation of an easier to fabricate, more resilient and thinner probe. This design also provides an within probe ground plane, electrical stimulation and pseudo-reference electrode. It will be appreciated that the use of a stainless steel wafer is a complement for dealing with optogenetics.

Polyimide flexible probes with simultaneous multiple recording sites detecting signals from individual neurons have been disclosed herein. Double sided flexible brain probes are built herein on a flexible wafer using all biocompatible materials and processes: polyimide—Kapton, acrylic—SU8, conformal coatings, Pt metallization, lift-off process.

While the above is a complete description of exemplary specific embodiments of the invention, additional embodiments are also possible. Thus, the above description should not be taken as limiting the scope of the invention, which is defined by the appended claims along with their full scope of equivalents.

We claim:

1. A method of fabricating a dual-sided biomorphic polymer-based microelectrode array, comprising:
spinning a lift-off layer (LOL) onto a flexible polymer wafer and pre-baking the LOL;
exposing the LOL to light in a mask aligner and etching to open areas for metal deposition;
conformally sputtering Pt onto the LOL, and subsequently lifting the Pt covered LOL to reveal Pt on polymer substrate on the flexible polymer wafer;
conformally spin coating polyimide layer onto the Pt on polymer substrate on the flexible polymer wafer, and pre-baking the wafer;
coating the wafer with photo-resist, baking the wafer, and exposing the wafer to light in a mask aligner; and
etching the flexible polymer wafer in a developer to open areas for etching polyimide and to finish formation of electrical insulating coating on conducting traces.

2. The method of claim 19, wherein the flexible polymer wafer is laminated Kapton polymer.
